(19) United States
(12) Patent Application Publication
(10) Pub. No.: US 2023/0200647 A1
(43) Pub. Date: Jun. 29, 2023

(54) METHOD FOR TRANSMITTING COMPRESSED BRAINWAVE PHYSIOLOGICAL SIGNALS

(71) Applicant: ExeBrain Co., Ltd., Taipei-City (TW)

(72) Inventors: Shu-Chun Kuan, Taipei City (TW); Jason Chun-Cheng Lin, Taipei City (TW); Chin-Yeh Lu, Taipei City (TW)

(21) Appl. No.: 17/893,456

(22) Filed: Aug. 23, 2022

(30) Foreign Application Priority Data
Dec. 29, 2021 (TW) 110149344

(51) Int. Cl. A61B 5/00; A61B 5/369; A61B 5/16

(52) U.S. Cl. CPC A61B 5/0006; A61B 5/369; A61B 5/7232; A61B 5/0022; A61B 5/165

(57) ABSTRACT

A method for transmitting compressed brainwave physiological signals is provided and including detecting a plurality of brainwave physiological signals of a subject, and generating an electroencephalography based on a time sequence of the plurality of brainwave physiological signals; splitting the electroencephalography into a plurality of sub-images based on the time sequence; using a plurality of static feature tags and a plurality of dynamic displacement tags stored in a brainwave database to identify at least one static feature tag and a plurality of associated dynamic displacement tags based on the time sequence according to the plurality of sub-images; generating at least one superimposed group tag, the superposed group tag is used to integrate the identified static feature tag and the associated dynamic displacement tag according to the time sequence; and transmitting the identified static feature tag, the associated dynamic displacement tag, and the superimposed group tag to a remote cloud system according to the time sequence.

### CROSS-REFERENCE TO RELATED APPLICATION

This application claims the priority of Taiwanese patent application No. 110149344, filed on Dec. 29, 2021, which is incorporated herewith by reference.

### BACKGROUND OF THE INVENTION

### 1. Field of the Invention

The present invention relates to a method for transmitting signals, and particularly to a method for transmitting compressed brainwave physiological signals.

### 2. The Prior Arts

Existing biofeedback training mainly uses a wireless device at the input terminal, such as a pair of electrode pads to compare the variation of brainwave before and after training in three regions of the parietal lobe, and a pair of electrode pads to detect the influence of neurofeedback for sensorimotor rhythm (SMR), or collect physiological signals and upload the physiological data to the cloud platform for analysis through wired or wireless transmission modules, the individual needs to open the APP or related applications to read data of the physiological device during the sleep period in a retrospective manner. However, in the prior art, users usually cannot obtain physiological information such as brainwaves or heartbeat variation immediately, and need to wait several hours to several days for interpretation.

Furthermore, the waveforms of physiological signals such as brainwaves consist of line segments composed of many points, so the original brainwaves are drawn by many points, and multiple points are drawn each second to form lines for different sampling rates, for example, the sampling frequency of 1000 means that there are 1,000 points to be drawn every second. If it is displayed on the X-Y plane, the X axis represents the recording time of brainwave, and the Y axis represents the potential difference and amplitude of the brainwave. If the original recording time of brainwave increases, the data and the difficulty in transmission also increases, thus it takes more time to achieve real-time comparison with the database.

Therefore, it is necessary to propose an improved method and system that can transmit a plurality of brainwave physiological signals with a large amount of data to the remote cloud system in real time, and users can adjust their own physiological signal for recovery through the visual or auditory feedback of the remote cloud system.

### SUMMARY OF THE INVENTION

In order to effectively solve the above problems, the present invention provides a method for transmitting compressed brainwave physiological signals is provided and including detecting a plurality of brainwave physiological signals of a subject, and generating an electroencephalography based on a time sequence of the plurality of brainwave physiological signals; splitting the electroencephalography into a plurality of sub-images based on the time sequence; using a plurality of static feature tags and a plurality of dynamic displacement tags stored in a brainwave database to identify at least one static feature tag and a plurality of associated dynamic displacement tags based on the time sequence according to the plurality of sub-images; generating at least one superimposed group tag, the superposed group tag is used to integrate the identified static feature tag and the associated dynamic displacement tag according to the time sequence; and transmitting the identified static feature tag, the associated dynamic displacement tag, and the superimposed group tag to a remote cloud system according to the time sequence.

The present invention further provides a transmission method for compressed brainwave physiological signals, which includes detecting a plurality of brainwave physiological signals of a subject, and generating an electroencephalography based on a time sequence of the plurality of brainwave physiological signals; using a plurality of feature tags and a plurality of index patterns stored in an brainwave database, and identifying a sequence of feature tags according to a plurality of electroencephalograms based on the time sequence; generating a biological feature sequence according to the identified sequence of feature tags and the time sequence wherein the biological feature sequence is composed of a plurality of index patterns, and the index pattern of the biological feature sequence is identified according to the identified sequence of feature tags; and transmitting a plurality of index patterns of the biological feature sequence to a remote cloud system according to the time sequence.

According to an embodiment of the present invention, the method for transmitting the compressed brainwave physiological signals uses a shape compression technique to compress the brainwave physiological signals through a static base value of screen and a displacement of screen of a difference between waveforms of different channels.

According to an embodiment of the present invention, a plurality of shape tags include: a static feature tag background-frame (referred to as B-Frame), an associated dynamic displacement tag movement-frame (referred to as M-Frame) and an superimposed group tag grouping-frame (referred to as G-Frame), the static feature tag is a static base value of the brainwave physiological signal, the associated dynamic displacement tag is a displacement of a signal value of the next frame, and the superimposed group tag is a message to deal with the static feature tag and the associated dynamic displacement tag.

According to an embodiment of the present invention, the plurality of brainwave physiological signals include power, frequency, current, current source density, asymmetry, coherence or phase lag.

According to an embodiment of the present invention, a plurality of index patterns are generated by training a neural network using a plurality of electroencephalograms, and each index pattern is represented by a combination of feature tags.

The transmission method of the present invention applied in the physiological signal long-distance bidirectional communication processing system can improve the evaluation efficiency, and the remote real-time feedback can be achieved in the biological feedback training system, so that the subject can immediately understand the condition and can adjust the physiological signals through the feedback for recovery.

### BRIEF DESCRIPTION OF THE DRAWINGS

FIG. 1 is a diagram showing the structure of the comparison feedback system for the transmission of compressed brainwave physiological signals from location A to location B.

FIG. 2 is a schematic diagram of two transmission processes of the compressed brainwave physiological signal transmission method of the present invention.

FIG. 3 and FIG. 4 are schematic diagrams of a compressed brainwave physiological signal transmission method using shape compression technology according to a first embodiment of the present invention.

FIG. 5 is a flowchart of a method for transmitting a compressed brainwave physiological signal according to the first embodiment of the present invention.

FIG. 6 and FIG. 7 are schematic diagrams of a method for transmitting a compressed physiological signal by an electroencephalogram pattern according to a second embodiment of the present invention.

FIG. 8 is a flowchart of a method for transmitting a compressed brainwave physiological signal according to the second embodiment of the present invention.

FIG. 9 and FIG. 10 are schematic diagrams of index patterns combined by different feature tags.

FIG. 11 is a schematic diagram of the behavioral performance or mental process corresponding to the biological sequences combined with different index patterns.

FIG. 12 is a schematic diagram of a compression and transmission method according to an embodiment of the present invention.

### DETAILED DESCRIPTION OF THE PREFERRED EMBODIMENT

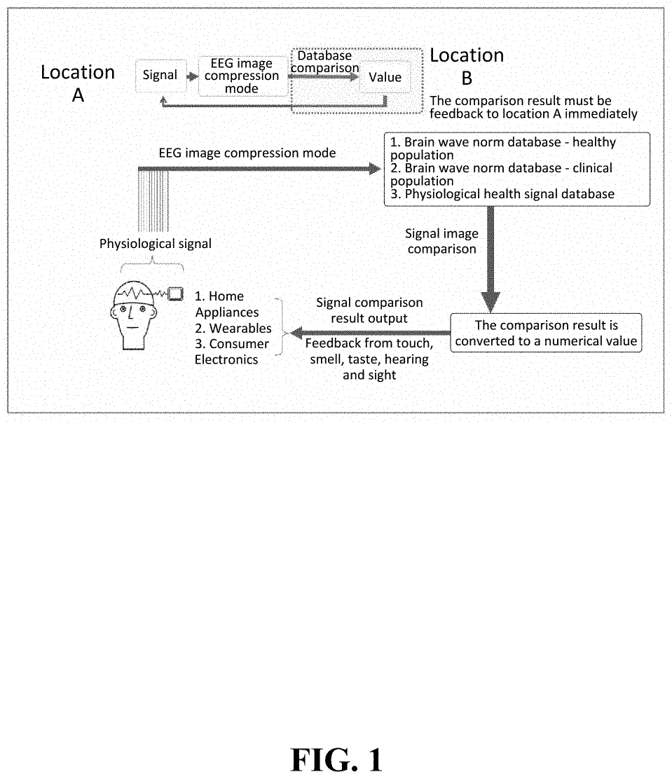

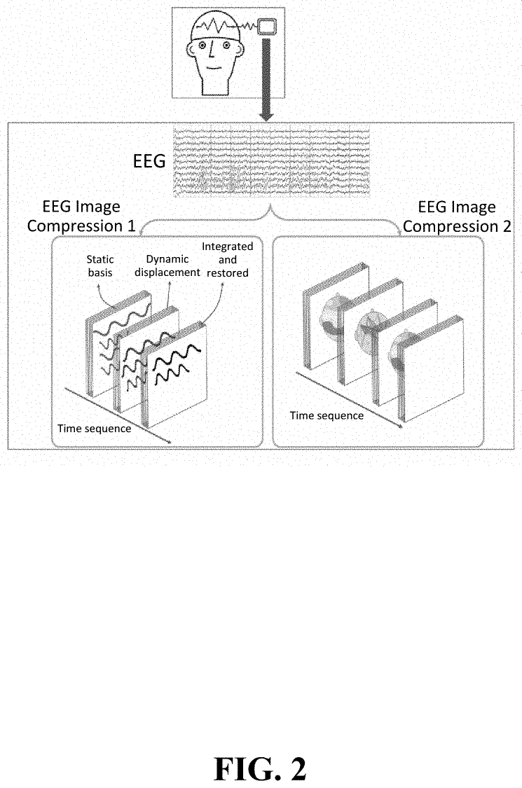

Please refer to FIG. 1 and FIG. 2, FIG. 1 is a diagram showing the structure of the comparison feedback system for the transmission of compressed brainwave physiological signals from location A to location B. The compression comparison method of the present invention is an electroencephalogram compression mode. FIG. 2 is a schematic diagram of two transmission processes of transmission method of the compressed brainwave physiological signal of the present invention. The present invention converts the original brainwave signal composed of dots into two kinds of picture images and performs dynamic comparison through the comparison of image compression mode.

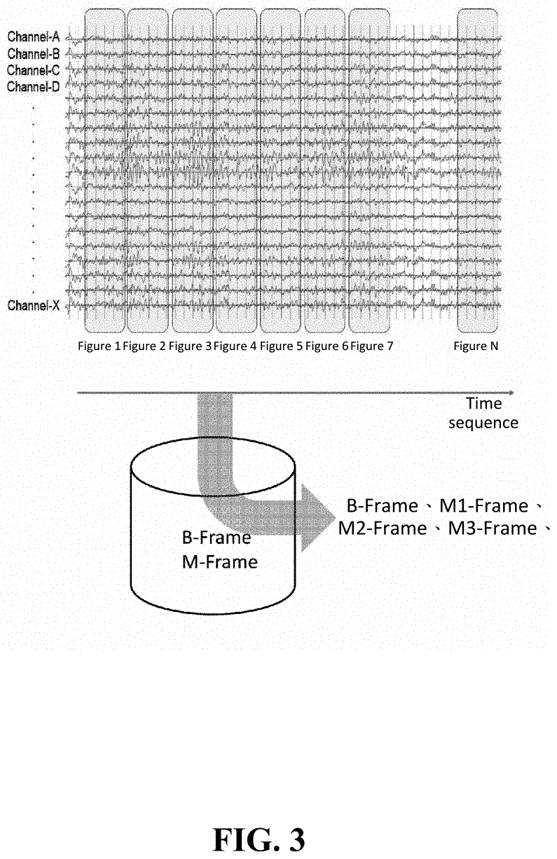

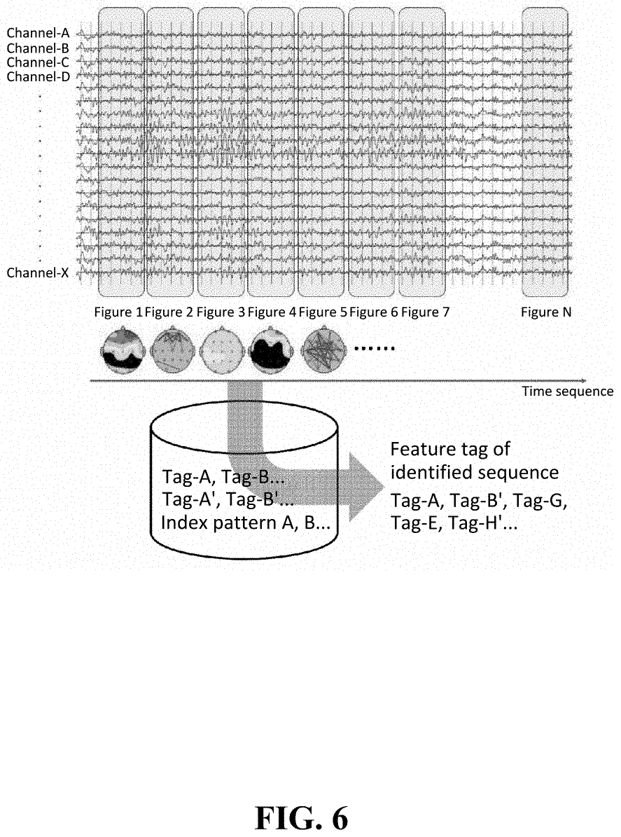

The two types of image processing of the present invention are: EEG image compression technology 1: dividing a plurality of brainwave physiological signals into a brainwave signal image file, and dividing the brainwave signal image based on a time sequence into a plurality of sub-images, and then identifies the plurality of sub-images as shape tags like static feature tags (B-Frame), associated dynamic displacement tags (M-Frame) and superimposed set tags (G-Frame). The brainwave physiological signals are transmitted through EEG image compression technology, please refer to FIG. 3 to FIG. 5. (2) EEG image compression technology: the original brainwave signals collected from different channels can be analyzed through algorithms to analyze the correlation of brainwaves between different points. The analysis of the correlation is like coherence, phase lag, power, asymmetry, etc., to generate complex electroencephalograms in time sequence, and then set feature tags for the complex electroencephalograms to generate a biological feature sequence, the biological feature sequence is composed of a plurality of index patterns, and the index pattern of the biological characteristic sequence is identified according to the feature tags of the calibration sequence, please refer to FIG. 6 to FIG. 12.

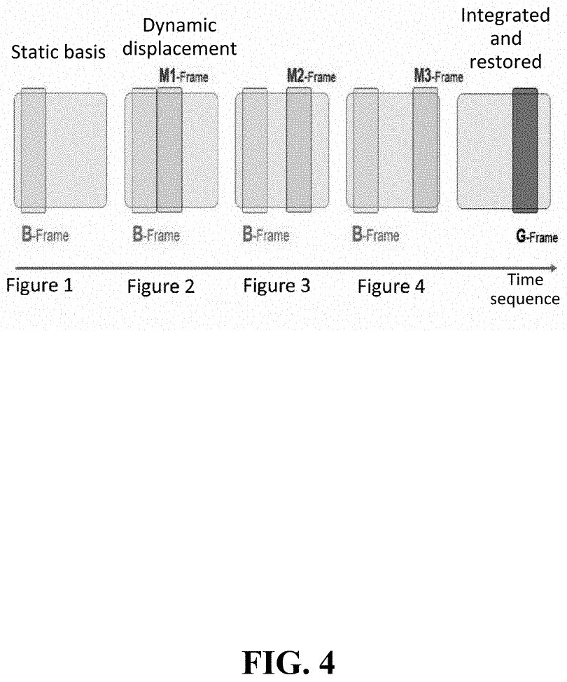

In the first embodiment of the present invention, please refer to FIG. 3 and FIG. 4, the brainwave physiological signals of different channels Channel-A to Channel-X are detected by using a brainwave collection device at home, such as a brainwave cap device used to measure a subject's EEG. The compression transmission method used in the present invention is to use EEG image compression technology 1: compressing the brainwave physiological signals through a static base value of screen and a displacement of screen of a difference between waveforms of different channels Channel-A to Channel-X. The collected brainwave physiological signals here are used to generate a EEG image, which is divided into a combination of multiple pictures (sub-pictures) FIG. 1 to Figure N in FIG. 3 in a fixed period of time, and each picture (sub-picture) is tagged. Referring to FIG. 4, after converting the complex brainwave physiological signals into a brainwave signal image file, the brainwave signal image is divided into a plurality of sub-images based on a time sequence, and shape tags such as the static feature tag background-frame (referred to as B-Frame), the dynamic displacement tag movement-frame (referred to as M-Frame) and the superimposed group tag grouping-frame (referred to as G-Frame), the static feature tag background-frame (referred to as B-Frame) is an image structure based on the background, the dynamic displacement tag movement-frame (M-Frame) is the difference value of the tagged image in the time sequence, and the superimposed group tag grouping-frame (G-Frame) is the background and the difference value.

For example, a piece of electroencephalogram will have a common static feature tag background-frame (B-Frame), and the background value is fixed over time. However, as time passes by, the change is associated dynamic displacement tag movement-frame (M-Frame), and the superimposed group tag grouping-frame (G-Frame) is the image processing static feature tag and dynamic displacement tag, so that different dynamic displacement tags (M1—Frame, M2—Frame, M3—Frame) and static feature tags (B-Frame) are integrated together. Such concept is similar to that animation is composed of different static panes, and dynamic effects are generated due to the rapid playback of the panes. The above three marking methods are to capture common static panes, dynamic displacement information that changes over time, and to provide the integrated dynamic and static tags. For example, in a video of a basketball player dribbling and dunking with the ball, the basket and the background are static images (B-Frame), the image of a basketball player dribbling to a dunk can be divided into different pictures. If the original signal is used for transmission, all the original static features (B-Frame) and dynamic displacement (M-Frame) are transmitted, which may easily result in excessive size of signal. In this mode, if the static features (B-Frame) are common feature value, then as long as the dynamic difference value of the dynamic displacement (M-Frame) is transmitted and the feature command of the integrating the superimposed set (G-Frame) is provided, the amount of transmitted information can be reduced, and the real-time data packet processing can be achieved without transmitting a lot of raw signals.

These tagging methods used by the method of the present invention use a brainwave database for tagging, and find out the characteristics of different behavioral performance and mental process through data analysis and calculation, and find out the combination of the static features (B-Frame), the dynamic displacement (M-Frame) and superimposed set (G-Frame) is stored in the brainwave database.

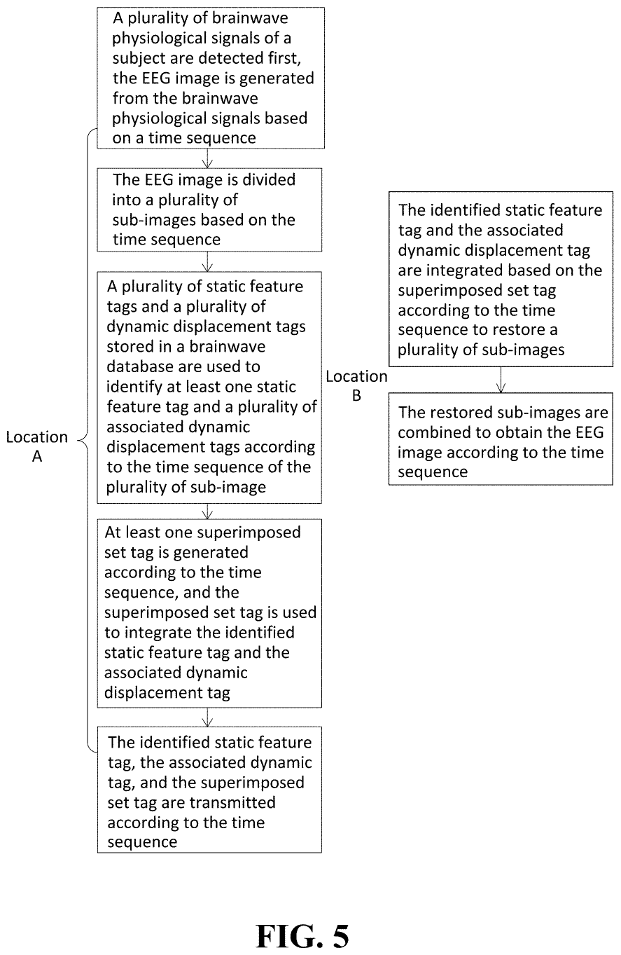

Please refer to FIG. 5 for the specific flowchart of the EEG image compression technology 1 used in the present invention. In location A (such as the user's home), a plurality of brainwave physiological signals of a subject are detected first, the EEG image is generated from the brainwave physiological signals based on a time sequence, the EEG image is divided into a plurality of sub-images; a plurality of static feature tags and a plurality of dynamic displacement tags stored in a brainwave database are used to identify at least one static feature tag B and a plurality of associated dynamic displacement tags M according to the time sequence of the plurality of sub-image; at least one superimposed set tag G is generated according to the time sequence, and the superimposed set tag is used to integrate the identified static feature tag B and the associated dynamic displacement tag M; the identified static feature tag B, the associated dynamic tag M, and the superimposed set tag G are transmitted according to the time sequence. In location B (such as a remote cloud system), the identified static feature tag B and the associated dynamic displacement tag M are integrated based on the superimposed set mark G according to the time sequence to restore a plurality of sub-images; the restored sub-images are combined to obtain the EEG image according to the time sequence.

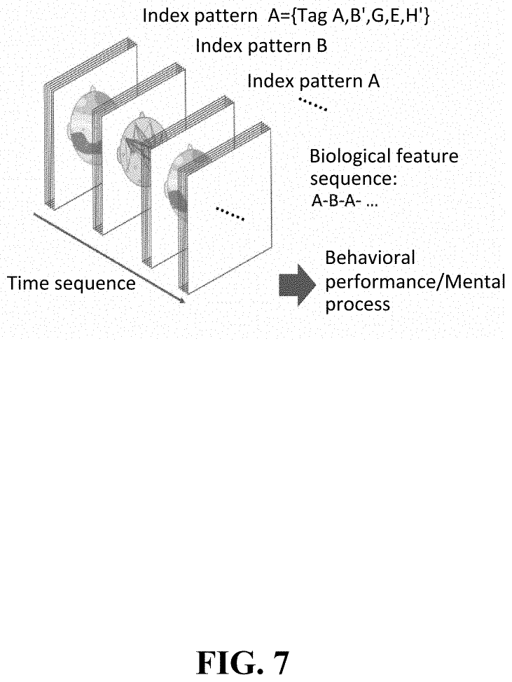

In the second embodiment of the present invention, please refer to FIG. 6 and FIG. 7, in the EEG image compression technology 2, for example of the power, frequency, current, current source density, asymmetry, coherence or phase lag of the brainwave collected from different channels of the brainwave cap, the relation of brainwaves between different channels can be analyzed through algorithms and include coherence, phase lag, power spectrum, asymmetry, etc.

The above-mentioned algorithm analysis includes, but not limited to, the use of Fourier transform, and the algorithm technology of beamforming can also be further added, and also includes other algorithms that can generate results. When the analysis of the Fourier transform is used to explain the analysis of the coherence, the EEG image comes from the power spectral density (PSD) of the EEG, and the analysis of the coherence is calculated from the electrode positions of X and Y, the densities Pxx(f) and Pyy(f) of each power spectrum, and the cross power spectral density Pxy(f) of X and Y. The frequency band 1-40 Hz is extracted from these EEG signals, and the electrode positions are at Delta(δ: 1-4 Hz), Theta(θ: 4-8 Hz), Alpha(α: 8-12 Hz), Beta(β: 13-30 Hz) and Gamma(γ: 30-40 Hz) will also perform analysis of coherence.

$${C}_{xy} ( f ) = \frac{{\vert {P}_{xy} ( f ) \vert}^{2}}{{P}_{xx} ( f ) \cdot {P}_{yy} ( f )}$$

References of the embodiment include: Unde, S. A., & Shriram, R. (2014). Coherence Analysis of EEG Signal Using Power Spectral Density. 2014 Fourth International Conference on Communication Systems and Network Technologies. doi:10.1109/csnt.2014.181; and Cao, Z., Lin, C.-T., Chuang, C.-H., Lai, K.-L., Yang, A. C., Fuh, J.-L., & Wang, S.-J. (2016). Resting-state EEG power and coherence vary between migraine phases. The Journal of Headache and Pain, 17(1). doi:10.1186/s10194-016-0697-7.

As shown in FIG. 7, each EEG image is tagged with feature tags Tag-A-Tag-Z and Tag-A′-Tag-Z′, and several feature tags can be combined to form a certain A specific index pattern, such as index pattern A-Z, index pattern A is composed of tag Tag-A, tag Tag-B′, tag Tag-G, tag Tag-E and tag Tag-H′, that is, index pattern A can be represented by the function f(Tag-A, Tag-B′, Tag-G, Tag-E, Tag-H′), and so on. The composition of different patterns will form indexes of specific behavioral performance or mental process.

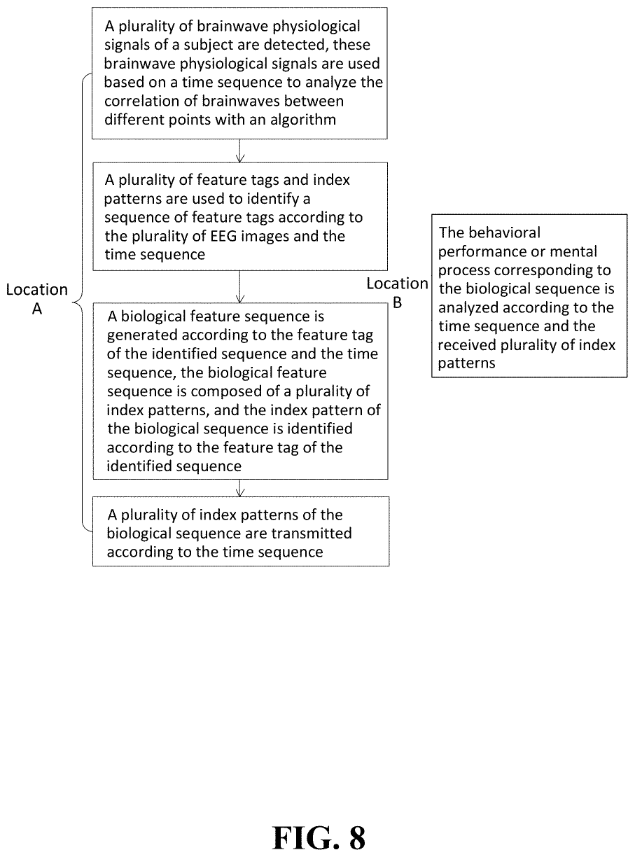

Please refer to FIG. 8 for the specific process of the EEG image compression technology 2 of the present invention. In location A (such as the user's home), a plurality of brainwave physiological signals of a subject are detected, these brainwave physiological signals are used based on a time sequence to analyze the correlation of brainwaves between different points with an algorithm, and identify a sequence of feature tags according to the plurality of EEG images and the time sequence; a biological feature sequence is generated according to the feature tag of the identified sequence and the time sequence, the biological feature sequence is composed of a plurality of index patterns, and the index pattern of the biological sequence is identified according to the feature tag of the identified sequence; a plurality of index patterns of the biological sequence are transmitted according to the time sequence. In location B (such as a remote cloud system), the behavioral performance or mental process corresponding to the biological sequence is analyzed according to the time sequence and the received plurality of index patterns.

The index pattern formed by the EEG image compression technology 2 of the present invention can be combined through an algorithm with the feature tags frequently used, and tagged as index pattern A, index pattern B, index pattern C, index pattern Z, etc. The sequence combined between the index patterns is the performance of a certain behavior and mental feature. FIG. 7 shows the combination of the index pattern A, the index pattern B, and the index pattern A, which can be composed of an EEG energy image (colored block graph) and an EEG correlation graph (line graph). The EEG correlation graph (line graphics) mainly uses Fourier transform, and can also be generated by adding beamforming algorithm technology. The EEG energy graph (colored block graph) is a potential line or an isoelectric line drawn according to the voltage potential generated by the neural current around each electrode.

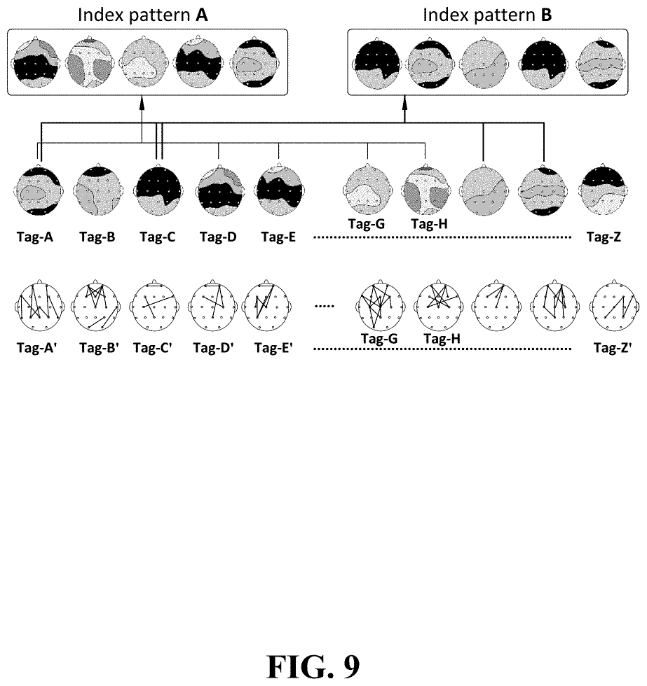

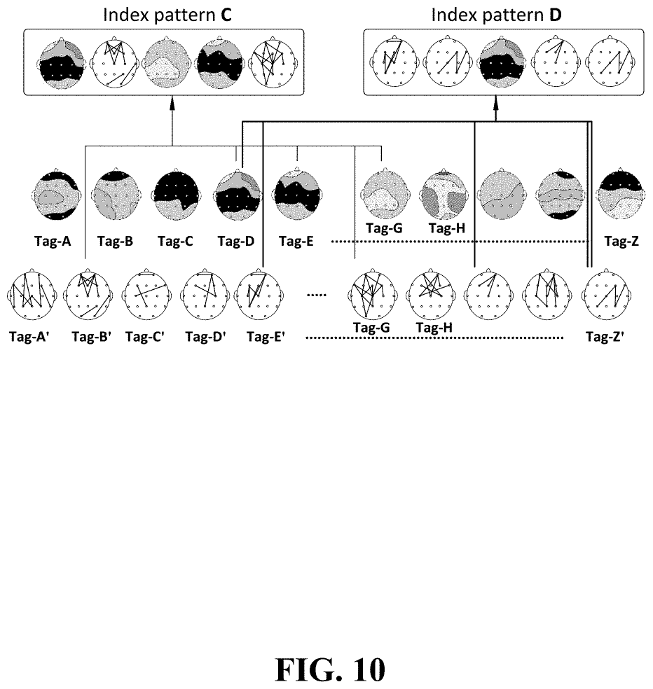

Please refer to FIG. 9 and FIG. 10, the index pattern A and index pattern B of FIG. 9 are composed of colored block graphics, and the index pattern C and index pattern D of FIG. 10 are composed of colored block graphics and line graphics, for example, the index pattern C is composed of Tag-B′ (line graph), Tag-D (colored block graph), Tag-E (colored block graph), and Tag-G (colored block graph). However, the composition of these index patterns is not static, but the dynamic changes generated by the time axis of Tag-B′, Tag-D, Tag-E, and Tag-G, which forms the index pattern C. The brainwave pattern features of these mental and behavioral features are formed by the brainwave database. For example, the brainwave database has multiple brainwave data related to insomnia. The index patterns and severity levels of these insomnia are classified, the index pattern combination of the sleep brainwave can be tagged through the morphological analysis, classification, grouping and feature extraction by the algorithm.

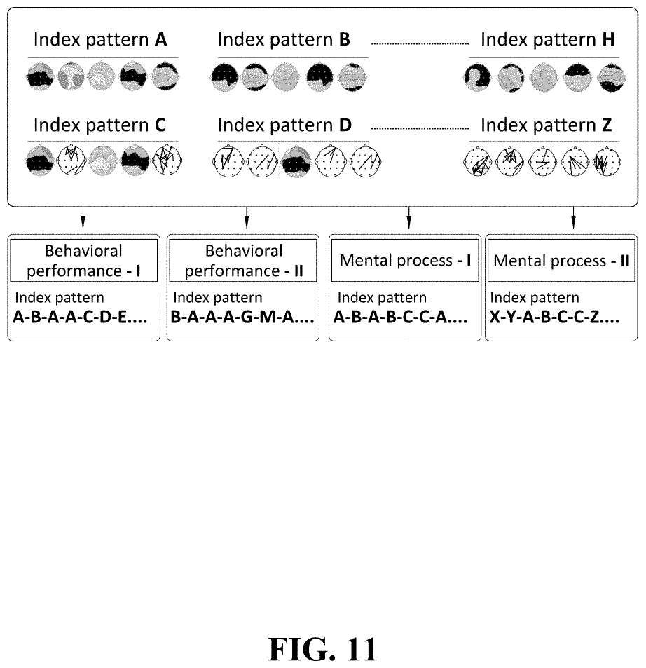

Please refer to FIG. 11. FIG. 11 is a schematic diagram of the behavioral performance or mental process corresponding to the biological sequences combined by different index patterns. Behavioral performance-I is represented by the index pattern A-B-A-A-C-D-E . . . and mental process-I is represented by the index pattern A-B-A-B-C-C-A . . . . Various behavioral performance and mental process can be represented by specific biological patterns. For example, the composition in FIG. 11 is similar to the expression of the gene sequence, the gene sequence presents the proper state of human body functions, and the gene mutation may cause pathological changes. However, genes are innately determined, and the composition of biological patterns referred to in this case is the biological feature sequence of behavioral performance or mental process, and the positioning through biological features (as shown in the example of the brainwaves) can be used to identify a certain behavioral performance or mental process of the mind. The neurofeedback is to recover through the adjustment mechanism of the subject, that is, to affect the physical or psychological state through physiology. Some simple behaviors or mental states may only require 1 to 3 patterns for identification, but for more complex behaviors or mental states, more patterns may be required for identification.

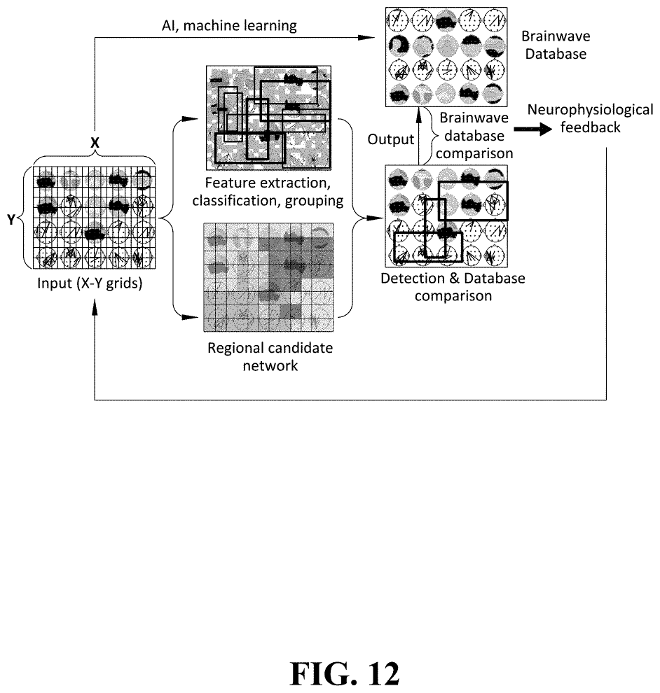

Please refer to FIG. 12, which is a schematic diagram of a compression and transmission method according to an embodiment of the present invention. The calculation and comparison method shown in FIG. 12 can achieve wireless long-distance transmission and maintain signal distortion. The method includes the following steps: obtaining a biological index data at the input terminal of the subject, and uploading the biological index data through a docking device to the remote cloud system for comparison with the database; dividing the biological data, and then performing feature extraction, classification and clustering, and region proposal networks (RPN) analysis to complete the comparison more accurately and quickly, and also provide neurofeedback to the region of interest (ROI) in the brain; the biological types in the database are compared after calculation and comparison, and the parameters related to the difference and similarity of the comparison are converted into feedback signals to the subject.

The two compression transmission methods of the present invention are used to transmit the EEG images generated by the brainwave physiological signals to a closed-loop loop system for remote cloud processing. The closed-loop loop system includes a subject terminal and a computing terminal.

In the closed-loop system, the subject terminal uses two compression transmission methods to compress and transmit the EEG images generated by the brainwave physiological signals to the computing terminal in the cloud, and then the computing terminal performs decompression. After obtaining the EEG images, the EEG images are compared with those stored in the database. Therefore, the comparison result is used to generate a feedback signal, and the feedback signal is returned from the computing terminal to the subject terminal.

The method for long-distance transmission of physiological signals used in the present invention includes the following steps: using a home brainwave collecting device such as a brainwave cap device at the subject terminal of the closed-loop system to generate brainwave physiological signals of different channels, equal signals form a EEG image for compression processing; the subject terminal transmits the information of the compressed EEG images to the computing terminal of the system; after decompression at the computing terminal, the information of the EEG image is obtained; the data of a brainwave database is used for comparison to generate a comparison result and a feedback signal, so as to reduce the data transmission between the computing terminal and the subject terminal. For example, when the system is used for biological feedback training, a biological index of the signal is compared with a detection database including brainwave and heart rate variability data to generate a comparison result and a feedback signal; and the computing terminal transmits the feedback signal to the subject. The time interval between the subject generating the signal and receiving the feedback signal is less than a threshold value. The threshold value is usually within 3 seconds, but can also be set as 5 seconds, 10 seconds, 20 seconds, 30 seconds depending on the requirement, and the threshold value is not limited to 3 seconds, but not exceeding 30 seconds.

In the method of the present invention, after the subject transmits the compressed signal, the computing terminal performs comparison with the brainwave database to generate a comparison result and the feedback signal, and then the feedback signal needs to be sent back to the subject. Meanwhile, the subject terminal continues to generate brainwaves or other physiological signals and continues to compress and upload them to the computing terminal, forming a closed-loop feedback mechanism. In the process of generating the signal, the subject terminal is also compressing the signal simultaneously, and also receiving the feedback signal for adjustment. Therefore, as to the technology for transmission, calculation, and comparison, the system and method of the present invention have higher transmission and comparison efficiency. The compression for transmission and comparison for feedback used in the present invention can provide the efficient feedback of the user's physiological signals, and efficient comparison and calculation of brainwaves, the possible brain regions and patterns included in the signals can be represented by trillions types of waveforms.

By using the comparison feedback method of the present invention in a closed-loop system for long-distance transmission of physiological signals, in addition to making the long-distance transmitted signal undistorted, the long-distance transmitted signal can be compared and returned. Technically complex physiological signals (such as EEG images) usually take a period of time for calculation, but the method of the present invention can transmit, compare and feedback more efficient, and the cost time must be within the allowable range (the goal of the present invention is to maintain the delay within 3 seconds though 30 seconds is also allowable), so the evaluation efficiency can be improved, and the remote real-time feedback can be achieved in the biological feedback training system, so that the subject can immediately understand the conditions and adjust through the physiological signals feedback for recovery.

The present invention is not limited to the above-described embodiments, and it will be clear to those skilled in the art that various modifications and changes can be made to the present invention without departing from the spirit or scope of the present invention.

Accordingly, this invention is intended to cover modifications and variations made to this invention or within the scope of the appended claims and the equivalents.

What is claimed is:

1. A method for transmitting compressed brainwave physiological signals, comprising:
detecting a plurality of brainwave physiological signals of a subject, and generating an electroencephalography based on a time sequence of the plurality of brainwave physiological signals;
splitting the electroencephalography into a plurality of sub-images based on the time sequence;
using a plurality of static feature tags and a plurality of dynamic displacement tags stored in a brainwave database to identify at least one static feature tag and a plurality of associated dynamic displacement tags based on the time sequence according to the plurality of sub-images;
generating at least one superimposed group tag, the superposed group tag is used to integrate the identified static feature tag and the associated dynamic displacement tag according to the time sequence; and
transmitting the identified static feature tag, the associated dynamic displacement tag, and the superimposed group tag to a remote cloud system according to the time sequence.

2. The method for transmitting compressed brainwave physiological signals of claim 1, wherein the static feature tag is a static base value of the brainwave physiological signal, and the dynamic displacement tag is a signal displacement of a sub-image.

3. The method for transmitting compressed brainwave physiological signals of claim 1, wherein the plurality of brainwave physiological signals include power, frequency, current, current source density, asymmetry, coherence or phase lag.

4. The method for transmitting compressed brainwave physiological signals of claim 1 further comprising: the remote cloud system integrates the identified static feature tag and the associated dynamic displacement tag according to the time sequence and the superimposed group tag to restore a plurality of sub-images; and the remote cloud system combines the restored sub-images to obtain the electroencephalography according to the time sequence.

5. A transmission method for compressed brainwave physiological signals, comprising:
detecting a plurality of brainwave physiological signals of a subject, and generating an electroencephalography based on a time sequence of the plurality of brainwave physiological signals;
using a plurality of feature tags and a plurality of index patterns stored in an brainwave database, and identifying a sequence of feature tags according to a plurality of electroencephalograms based on the time sequence;
generating a biological feature sequence according to the identified sequence of feature tags and the time sequence wherein the biological feature sequence is composed of a plurality of index patterns, and the index pattern of the biological feature sequence is identified according to the identified sequence of feature tags; and
transmitting a plurality of index patterns of the biological feature sequence to a remote cloud system according to the time sequence.

6. The transmission method of claim 5 further comprising: according to the time sequence, the remote cloud system analyzes behavioral performance or mental process corresponding to the biological feature sequence according to the plurality of index patterns received.
